(19) United States
(12) Patent Application Publication
(10) Pub. No.: US 2004/0010377 A1
(43) Pub. Date: Jan. 15, 2004

(54) Clustering method

(76) Inventors: Mark Swindells, Lincs (GB); Mark Rae, London (GB)

Correspondence Address: DARBY & DARBY P.C., P. O. BOX 5257, NEW YORK, NY, 10150-5257, US

(21) Appl. No.: 10/221,834

(22) PCT Filed: Mar. 14, 2001
(86) PCT No.: PCT/GB01/01120

(30) Foreign Application Priority Data
Mar. 14, 2000 (GB) 0006150.7

(51) Int. Cl. G06F 19/00; G01N 33/48; G01N 33/50

(52) U.S. Cl. 702/20

(57) ABSTRACT

The invention relates to a method for reducing the number of results generated by the alignment of a query protein or nucleotide sequence against a target protein or nucleotide sequence by an alignment algorithm, the method comprising the step of combining two or more alignment results into a single alignment result for each specific region of sequence alignment identified between query and target sequences.

The invention relates to a method for reducing the number of alignments generated between protein-or nucleotide sequences.

All cited documents are incorporated herein in their entirety.

There has recently been an unprecedented increase in the rate of generation of sequence data, due to advances in genetics and molecular biology and to the advent of large scale sequencing projects. Many experimental techniques needed to accelerate the generation of sequence data on a large scale have now been successfully scaled-up, allowing these strategies to be transported from the laboratory bench into an industrial context. In this environment, these techniques involve minimal human intervention and allow very rapid sequencing to take place at a relatively low cost.

As a result, over the last ten years, the volume of sequence data has continued to double every 18 months and this increase shows no sign of slowing pace. A significant increase in the early 1990s was associated with the deposit of tranches of Expressed Sequence Tags (ESTs). The sequence information generated so far comes from a diverse selection of organisms. The main source of large deposit is now for completed microbial organisms or large regions of eukaryotic chromosomes.

The amount of detail contained in sequence databases such as GenBank (http://www.ncbi.nlm.nih.gov), the EMBL nucleotide data library at the European Bioinformatics Institute (http://www.ebi.ac.uk) and the DNA database of Japan (DDBJ) at the National Institute of Genetics (http://www.ddbj.nig.acjp), is immense and can cover such diverse information as the origin of the organism or chromosome from which the sequence data are derived and intron/exon information for each gene. The protein coding regions for each stretch of sequence of DNA may also be given (whether predicted or experimental).

Databases such as Swissprot (http://expasy.hcuge.ch/) and PIR (http://pir.georgetown.edu/) devote themselves solely to protein sequence data. These databases also contain elements of additional information and include details such as the presence of N-terminal secretory signals, membrane-spanning regions and regions with other atypical residue compositions.

As the number of sequence entries continues to rise, there is a concomitant increase in the number of the database sequence entries that are related. Homologous genes may occur in the same organism or in different organisms. The degree of similarity may range from low amino acid identity to high or even total identity. The latter happens when several groups have submitted the same sequence.

Sequence redundancy is both a blessing and a problem. It provides an ability to build a profile that describes the most pertinent points of a homologous family, by iteratively searching the database and refining the profile to identify progressively more distant relationships. However, the iterative process generally means that relationships identified earlier in the search can and usually appear in subsequent iterations.

An alignment program such as the Position Specific Iteration Basic Local Alignment of Sequences Tool (PSI-BLAST) (Nucleic Acids Res 1997 September 1;25(17):3389-3402), is a typical example of an algorithm where a large number of repeating sequence hits are generated. In this particular case, although the alignment and E-Value (expectation value) may change between iterations, the alignment may still describe the same basic region of similarity between the two sequences. Other algorithms, such as the Blast program, may also generate multiple overlapping results. Here, although there is no iteration, we may still get multiple overlapping results. Of course, there may be more than one non-overlapping similarity, in the case of a multi-domain protein and this should also be taken into account what removing redundancy.

There is thus a great need in the art for an effective method to combine multiple results from sequence alignments into a single result for each region of similarity identified.

### SUMMARY OF THE INVENTION

Accordingly, the present invention provides a method for reducing the number of results generated by the alignment of a query sequence against a target sequence by an alignment algorithm, said method comprising the step of combining two or more alignment results into a single alignment result for each specific region of sequence alignment identified between the query and target sequences.

Preferably, the method is a computer-implemented method.

As a starting point for the method, alignment results are obtained from an alignment algorithm such as BLAST (Altschul et al., (1990) J Mol Biol, 215: 403-410); PSI-BLAST (Altschul et al., (1997) NAR, 25(17): 2289-2302); FASTA (Pearson & Lipman, (1988) Proc Natl Acad Sci USA; 85(8): 2444-8), Smith-Waterman (Smith and Waterman, (1981) J Mol Biol, 147: 195-197); and Needleman and Wunsch, (1970) J Mol Biol, 48: 443-453). For each region of alignment between two sequences, such programs may output a number of results that represent overlapping alignments in the same region. The aim of the method of the invention is to reduce the number of these results or “hits”, since many of the hits in fact represent minor variants of the same alignment.

The method of the invention has been found to be particularly effective in significantly reducing the number of alignments generated by PSI-BLAST. The invention is particularly applicable to iterative alignment methods such as PSI-BLAST since these programs tend to generate a large number of results for a single alignment of two sequences. In a typical alignment of two sequences, the number of sequence “hits” processed using a method according to the invention may be reduced to as little as one fiftieth of their original number.

Other non-iterative alignment algorithms may also generate more than one result for the alignment of two sequences. For example, multiple pairwise alignments of two sequences could be run using the Smith-Waterman algorithm using variations in the parameter settings for each pairwise alignment. In an alternative scenario, different scoring matrices could be used for each pairwise alignment.

In one aspect of the invention, there is provided a computer-implemented method for reducing the number of results generated by the alignment of a query sequence against a target sequence by an alignment algorithm, said method comprising the steps of:

(a) extracting said alignment results;

(b) combining two or more alignment results into a single alignment result for each specific region of sequence alignment identified between query and target sequences; and

(c) outputting said single result.

The principle of the method of the invention is outlined in further detail below.

When two sequences are aligned, regardless of the algorithm used, the resultant values can be split into two groups.

The first group contains those values that describe the location of the aligned region of the two sequences denoted A & B. These results can always be represented by four numbers, as gaps in the alignment are not taken into consideration.

The first two numbers of the first group describe the extent of the aligned region on sequence A, denoted as [F_{A}, T_{A}], and the second two describe the extent of the aligned region on sequence B, denoted by [F_{B}, T_{B}]

The second group contains those output values which are related to the score or scores produced by the alignment algorithm. For example, useful outputs from the PSI-BLAST algorithm include the E-Value and the iteration number.

To explain the rationale governing the decision as to whether or not any two alignments are combined into one, the representation shown in FIG. 1 may be used.

The horizontal axis represents the residue numbers from sequence A, and the vertical axis residue numbers from sequence B. It can be seen that if perpendicular lines are drawn from the position of four numbers representing the alignment, then that alignment region is represented by a rectangle:

In considering two alignments, and whether or not they can be combined into one, there are three possible cases.

In the first case (FIG. 2), the two regions are disjoint, and so the two alignments can be trivially rejected as candidates for being combined.

In the second case (FIG. 3), one region is completely enclosed within another. These two alignments are therefore suitable for merging, with the new representative being the larger of the two regions.

Finally there is the case where the two regions intersect (FIG. 4). The method of the invention decides whether or not these two regions should be merged, based on the area of the intersection. If this area is significant, then the two alignments are merged into one.

The threshold value that defines a significant overlap varies depending on the algorithm or method that is being used to generate the alignment. Using PSI-BLAST alignment results, a figure of 90% has been found to work well (if the area of intersection of the two regions is greater than or equal to 90% of the area of the smaller of the two regions, then the regions are merged).

The value of 90% can of course be varied to suit the particular requirements of the analysis being carried out, but this figure was chosen as it worked well for the combination of results generated by PSI-BLAST. However, this figure is an arbitrary value that can be modified by a user depending upon the algorithm that is used. Preferably, this value is set between 80 and 99%, more preferably, between 85 and 95%.

If the two regions are suitable for merging, then the combined region then becomes the bounding box of the two rectangles (represented by the dashed line in FIG. 4).

For separate alignments of two sequences, the method of the invention can be illustrated as follows. As discussed above, a first alignment between a query sequence A at positions [F_{A}, T_{A}] and a target sequence B at positions [F_{B}, T_{B}] may be represented graphically with the horizontal axis representing the residue numbers from sequence A, and the vertical axis representing the residue numbers from sequence B, such that a rectangular region marked by co-ordinates [F_{A}, F_{B}], [T_{A}, F_{B}], [T_{B}, F_{A}], and [T_{A}, T_{B}] represents a first region of alignment. A second alignment between the query sequence at positions [F′_{A}, T′_{A}] and the target sequence at positions [F′_{B}, T′_{B}] may also be represented graphically such that a rectangular region marked by co-ordinates [F′_{A}, F′_{B}], [T′_{A}, F′_{B}], [T′_{B}, F′_{A}], and [T′_{A}, T′_{B}] represents a second region of alignment. According to the invention, the first and second alignments are combined if there is a significant region of intersection between the two regions of alignment.

Preferably, the two regions are combined if the area of intersection of the two regions is greater than or equal to 80% of the area of the smaller of the two regions. More preferably, this value is set at between 85 and 99%, more preferably, between 85 and 95%.

In the case where there are multiple alignment regions, such as when there is one alignment generated from each iteration of a repeating algorithm such as PSI-BLAST, the above calculations must be repeatedly performed, continually merging alignments together until no more candidates for merging are found. Finally there will then be one alignment representative for each distinct alignment region of the sequences that can be found.

The method may thus be broken down into steps involving extracting the results of the alignment of two separate sequences using a repeating alignment algorithm, followed by merging the results together if there is a significant region of overlap between them.

In order to perform this procedure efficiently, a ‘subset construction’ algorithm may be used (see, for example, Object-Oriented Software Construction, Bertrand Meyer [ISBN: 0136291554]). This will minimise the number of comparisons that need to be done between alignment pairs.

It should be noted that the example shown in FIG. 2, in which one region is completely enclosed by another, has been shown as a completely separate case. However in reality, this is just a special case of two regions intersecting, in which the area of overlap must be greater than a certain proportion (for example, 90%) of the smaller rectangle. The reason for showing this example as a separate case is that it is much easier to calculate than the general case of partial overlap. Therefore, if all of the enclosed alignments are removed first, there are less alignments to compare afterwards. This has the effect of speeding up the calculation. Accordingly, in the method of the invention, the step of merging alignment results together is preferably performed in iterative steps, whereby each alignment that is completely subsumed by another alignment is merged with the larger alignment before overlapping alignments are considered.

This aspect of the invention therefore provides a method according to any one of the aspects described above, wherein said combining step comprises the sequential steps of:

i. combining alignment regions in which one alignment region subsumes another; and

ii. combining alignment regions that only partially overlap.

It should be noted that alignment values are independent of the merging procedure and can be changed to suit the particular application. In the case of merging results from PSI-BLAST, the values that have been found to be of particular interest were the iteration number and the E-Value combination. These were required for the first, best and last iterations in which an alignment occurred.

In a particularly preferred embodiment of the invention, when two regions are merged using the above criteria, the lowest and highest iteration/E-Value pair present in the two alignments are stored in the combined alignment, along with the lowest E-Value achieved by either of the two alignments together with the iteration number at which this was achieved.

In use, it has been found that the application of this algorithm to the results of a PSI-BLAST search which ran for 20 iterations can reduce the total number of hits to as little as one fiftieth of their original number.

One example of the use of the method of the invention to reduce the number of alignments generated by an iterative alignment search is provided in co-pending co-owned United Kingdom patent application entitled “Database”. This application is directed to the generation of a non-redundant database of protein sequences. The relationship of every sequence to every other sequence in the database has been pre-calculated with exceptional sensitivity and reliability, using sophisticated algorithms, including sequence alignment algorithms. This necessitates the calculation and storage of around 100 million relationships. This task has been made considerably simpler by reducing the number of hits identified by performing multiple alignments on each of the sequences contained in the database before the calculation of relationships is performed. This has reduced the load of comparisons that must be performed in order to compile the database.

In a preferred embodiment of the invention, the method is performed to reduce the number of results generated by an iterative alignment search of sequences in a non-redundant database. This further reduces the load of comparisons that need to be performed when calculating relationships between proteins of differing sequence. A non-redundant database is a database in which identical or similar entries have been eliminated from the data resource, such that only a single entry remains for each sequence.

In a further embodiment of the invention, the results generated by this method may be output to include details such as the total number of iterations that an alignment algorithm such as PSI-BLAST or blastpgp performed and then, for each query sequence, a (merged group of) hit(s), optionally as space-separated columns, details may be selected from the following:

1. The name of the sequence hit.

2. The local hit number (such that this, grouped with the name of the sequence hit, are unique for a subject sequence).

3. The length of the match. This is the length of the longest match in the cluster.

4. The bit score of the hit with the “best” E-value.

5. The hit “E-value”: a normalization of the “bit score”, representing the confidence of the hit. This is the best (lowest) E-value over all the hits grouped.

6. The identical residues count of the hit with the “best” E-value.

7. The positive scores count of the hit with the “best” E-value.

8. The lowest index of the starting residue of the matches in the cluster in the subject sequence.

9. The highest index of the ending residue of the matches in the cluster in the subject sequence.

10. The lowest index of the starting residue of the matches in the cluster in the subject sequence.

11. The highest index of the ending residue of the matches in the cluster in the subject sequence.

12. The DNA match frame.

13. The lowest PSI-BLAST iteration of the hits in the cluster.

14. The evalue of the hit of the lowest PSI-BLAST iteration in the cluster.

15. The highest PSI-BLAST iteration of the hits in the cluster.

According to a further aspect of the invention, there is provided a computer apparatus adapted to reduce the number of results generated by the alignment of a query sequence against a target sequence, said apparatus comprising:

a processor means;

a memory means; and

computer software stored in said memory means and adapted to reduce the number of results generated by the alignment of a query sequence against a target sequence using a method according to any one of the aspects of the invention discussed above and output a single alignment result.

In a still further embodiment of the invention, there is provided a computer system adapted to reduce the number of results generated by the alignment of a query sequence against a target sequence, wherein said system performs a method as discussed above and outputs an alignment result.

Such a system may preferably comprise a central processing unit; an input device for inputting requests; an output device; a memory (at least one bus connecting the central processing unit, the memory, the input device and the output device); the memory storing a module that is configured so that upon receiving a request to align a query sequence with a target sequence, it performs a method according to any one of the aspects of the invention outlined above.

In the apparatus and systems of these embodiments of the invention, data may be input by downloading the sequence data from a local site such as a memory or disk drive, or alternatively from a remote site accessed over a network such as the internet. The sequences may be input by keyboard, if required.

The generated alignment may be output in any convenient format, for example, to a printer, a word processing program, a graphics viewing program or to a screen display device. Other convenient formats will be apparent to the skilled reader.

The means adapted to align said plurality of protein or nucleic acid sequences will preferably comprise computer software means. As the skilled reader will appreciate, once the novel and inventive teaching of the invention is appreciated, any number of different computer software means may be designed to implement this teaching.

The invention also provides a computer program product for use in conjunction with a computer, said computer program comprising a computer readable storage medium and a computer program mechanism embedded therein, the computer program mechanism comprising a module that is configured so that upon receiving a request to align two or more sequences together, it performs any one of the methods outlined above and outputs an alignment result.

The invention will now be described by way of example with particular reference to a specific algorithm that implements the process of the invention. As the skilled reader will appreciate, variations from this specific illustrated embodiment are of course possible without departing from the scope of the invention.

### BRIEF DESCRIPTION OF THE FIGURES

FIG. 1 shows a graphical representation of the region of alignment between two related sequences.

FIG. 2 shows the situation when the two alignment regions are disjoint.

FIG. 3 shows the situation when one region of alignment is completely enclosed by another.

FIG. 4 shows the situation when two regions of alignment intersect.

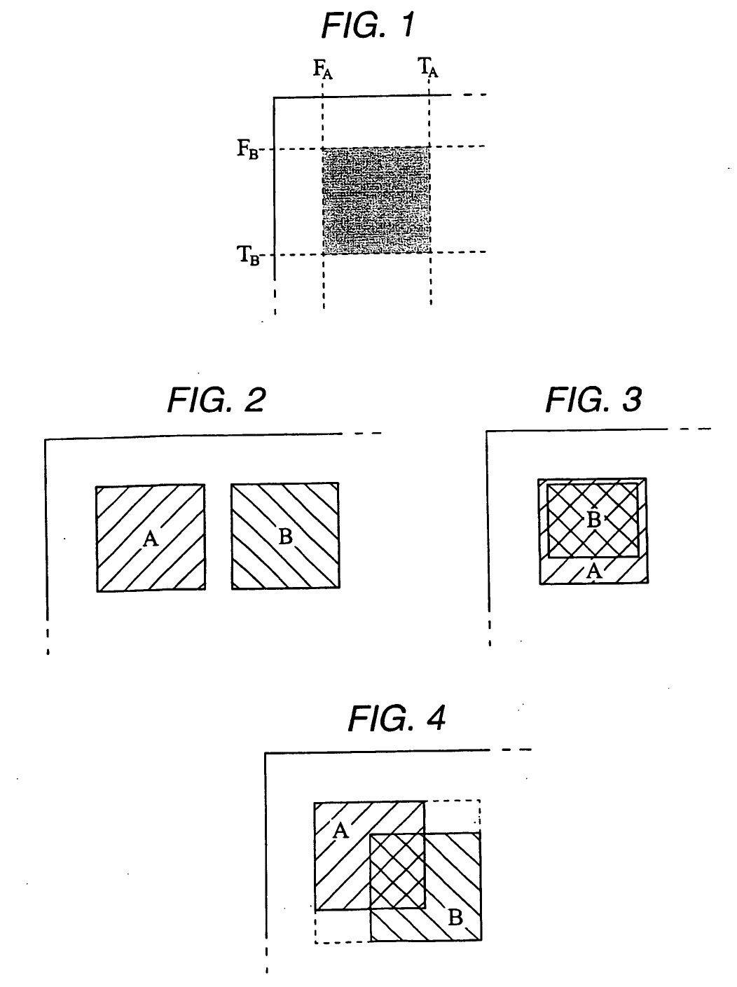

### EXAMPLE

The following is an example of the clustering procedure performed on a set of results produced by searching a sequence database using PSIBlast for the 1bh3 PDB protein sequence.

Below is a subset of the results from the PSIBlast search.

| Sequence | Length | Bit-Score | E-Value | ID | +ve | From | To | From | To | Iteration |
|---|---|---|---|---|---|---|---|---|---|---|
| gb\|g2853297 | 338 | 80.4 | 1e−14 | 10 | 20 | 1 | 283 | 5 | 320 | 5 |
| gb\|g2853297 | 338 | 78.0 | 6e−14 | 5 | 14 | 7 | 263 | 61 | 321 | 5 |
| gb\|g2853297 | 338 | 71.4 | 6e−12 | 8 | 16 | 10 | 257 | 38 | 334 | 5 |
| gb\|g2853297 | 338 | 75.3 | 4e−13 | 9 | 17 | 28 | 242 | 111 | 333 | 5 |
| gb\|g2853299 | 441 | 85.0 | 4e−16 | 8 | 20 | 2 | 283 | 51 | 335 | 5 |
| gb\|g2853299 | 441 | 91.3 | 6e−18 | 8 | 20 | 4 | 284 | 75 | 357 | 5 |
| gb\|g2853299 | 441 | 83.5 | 1e−15 | 9 | 20 | 5 | 285 | 142 | 424 | 5 |
| gb\|g2853299 | 441 | 79.2 | 3e−14 | 12 | 23 | 8 | 283 | 111 | 388 | 5 |
| gb\|g2853299 | 441 | 74.5 | 6e−13 | 12 | 27 | 29 | 288 | 40 | 300 | 5 |
| gb\|g2853297 | 338 | 122.0 | 2e−27 | 10 | 20 | 1 | 289 | 5 | 314 | 6 |
| gb\|g2853297 | 338 | 102.0 | 2e−21 | 8 | 20 | 3 | 255 | 65 | 334 | 6 |
| gb\|g2853297 | 338 | 59.7 | 2e−08 | 7 | 18 | 121 | 285 | 1 | 192 | 6 |
| gb\|g2853297 | 338 | 61.2 | 6e−09 | 8 | 23 | 132 | 290 | 1 | 172 | 6 |
| gb\|g2853299 | 441 | 88.1 | 5e−17 | 7 | 21 | 1 | 192 | 248 | 439 | 6 |
| gb\|g2853299 | 441 | 111.0 | 6e−24 | 9 | 22 | 2 | 286 | 51 | 337 | 6 |
| gb\|g2853299 | 441 | 107.0 | 9e−23 | 8 | 23 | 3 | 256 | 187 | 437 | 6 |
| gb\|g2853299 | 441 | 119.0 | 2e−26 | 8 | 18 | 3 | 289 | 136 | 420 | 6 |
| gb\|g2853299 | 441 | 125.0 | 4e−28 | 8 | 20 | 3 | 285 | 140 | 424 | 6 |
| gb\|g2853299 | 441 | 127.0 | 1e−28 | 10 | 21 | 4 | 287 | 75 | 369 | 6 |
| gb\|g2853299 | 441 | 113.0 | 1e−24 | 10 | 21 | 5 | 291 | 112 | 414 | 6 |
| gb\|g2853299 | 441 | 113.0 | 1e−24 | 7 | 20 | 5 | 273 | 168 | 437 | 6 |
| gb\|g2853299 | 441 | 114.0 | 6e−25 | 8 | 19 | 5 | 285 | 97 | 390 | 6 |
| gb\|g2853299 | 441 | 104.0 | 6e−22 | 7 | 21 | 10 | 289 | 49 | 325 | 6 |
| gb\|g2853299 | 441 | 108.0 | 4e−23 | 11 | 25 | 13 | 289 | 18 | 301 | 6 |
| gb\|g3876860 | 1805 | 62.8 | 2e−09 | 9 | 16 | 1 | 283 | 1040 | 1350 | 6 |
| gb\|g3876860 | 1805 | 70.6 | 1e−11 | 10 | 21 | 3 | 287 | 740 | 1033 | 6 |
| gb\|g3876860 | 1805 | 58.9 | 3e−08 | 7 | 17 | 4 | 224 | 1140 | 1380 | 6 |
| gb\|g3876860 | 1805 | 63.9 | 1e−09 | 11 | 21 | 4 | 292 | 446 | 763 | 6 |
| gb\|g3876860 | 1805 | 79.9 | 2e−14 | 9 | 18 | 4 | 289 | 836 | 1156 | 6 |
| gb\|g3876860 | 1805 | 63.6 | 1e−09 | 11 | 22 | 5 | 288 | 1010 | 1316 | 6 |
| gb\|g3876860 | 1805 | 72.9 | 2e−12 | 10 | 19 | 5 | 287 | 906 | 1202 | 6 |
| gb\|g3876860 | 1805 | 74.8 | 5e−13 | 12 | 22 | 10 | 285 | 973 | 1257 | 6 |
| gb\|g3876860 | 1805 | 85.8 | 3e−16 | 10 | 17 | 16 | 288 | 800 | 1077 | 6 |
| gb\|g3876861 | 1797 | 62.8 | 2e−09 | 9 | 16 | 1 | 283 | 1040 | 1350 | 6 |
| gb\|g3876861 | 1797 | 70.6 | 1e−11 | 10 | 21 | 3 | 287 | 740 | 1033 | 6 |
| gb\|g3876861 | 1797 | 58.9 | 3e−08 | 7 | 17 | 4 | 224 | 1140 | 1380 | 6 |
| gb\|g3876861 | 1797 | 63.9 | 1e−09 | 11 | 21 | 4 | 292 | 446 | 763 | 6 |
| gb\|g3876861 | 1797 | 79.9 | 2e−14 | 9 | 18 | 4 | 289 | 836 | 1156 | 6 |
| gb\|g3876861 | 1797 | 63.6 | 1e−09 | 11 | 22 | 5 | 288 | 1010 | 1316 | 6 |
| gb\|g3876861 | 1797 | 72.9 | 2e−12 | 10 | 19 | 5 | 287 | 906 | 1202 | 6 |
| gb\|g3876861 | 1797 | 74.8 | 5e−13 | 12 | 22 | 10 | 285 | 973 | 1257 | 6 |
| gb\|g3876861 | 1797 | 85.8 | 3e−16 | 10 | 17 | 16 | 288 | 800 | 1077 | 6 |
| gb\|g435535 | 235 | 55.8 | 3e−07 | 5 | 17 | 4 | 120 | 103 | 224 | 6 |
| gb\|g435535 | 235 | 73.7 | 1e−12 | 8 | 17 | 5 | 159 | 55 | 224 | 6 |
| gb\|g435535 | 235 | 79.5 | 2e−14 | 9 | 20 | 23 | 175 | 54 | 224 | 6 |
| gb\|g435535 | 235 | 78.0 | 6e−14 | 5 | 15 | 52 | 210 | 54 | 224 | 6 |
| gb\|g435535 | 235 | 78.0 | 6e−14 | 8 | 20 | 64 | 221 | 54 | 224 | 6 |
| gb\|g435535 | 235 | 83.0 | 2e−15 | 5 | 17 | 70 | 231 | 54 | 224 | 6 |
| gb\|g435535 | 235 | 84.2 | 8e−16 | 10 | 18 | 98 | 259 | 54 | 224 | 6 |
| gb\|g435535 | 235 | 80.7 | 9e−15 | 5 | 15 | 131 | 291 | 54 | 224 | 6 |
| gb\|g160473 | 280 | 56.2 | 2e−07 | 7 | 18 | 5 | 108 | 65 | 170 | 7 |
| gb\|g160473 | 280 | 58.5 | 4e−08 | 7 | 23 | 5 | 130 | 48 | 168 | 7 |
| gb\|g160473 | 280 | 60.1 | 1e−08 | 6 | 21 | 7 | 209 | 15 | 204 | 7 |
| gb\|g160473 | 280 | 83.8 | 1e−08 | 5 | 25 | 90 | 259 | 23 | 190 | 7 |
| gb\|g160473 | 280 | 64.4 | 7e−10 | 7 | 21 | 115 | 267 | 17 | 174 | 7 |
| gb\|g160473 | 280 | 66.3 | 2e−10 | 7 | 21 | 165 | 287 | 48 | 174 | 7 |
| gb\|g160473 | 280 | 50.7 | 9e−06 | 5 | 16 | 174 | 291 | 45 | 162 | 7 |
| gb\|g2853297 | 338 | 139.0 | 1e−32 | 8 | 17 | 1 | 292 | 5 | 303 | 7 |
| gb\|g2853297 | 338 | 118.0 | 5e−26 | 8 | 19 | 2 | 255 | 61 | 334 | 7 |
| gb\|g2853297 | 338 | 127.0 | 7e−29 | 6 | 17 | 5 | 291 | 1 | 311 | 7 |
| gb\|g2853299 | 441 | 126.0 | 1e−28 | 6 | 18 | 1 | 290 | 28 | 326 | 7 |
| gb\|g2853299 | 441 | 144.0 | 5e−34 | 8 | 20 | 1 | 291 | 139 | 433 | 7 |
| gb\|g2853299 | 441 | 139.0 | 2e−32 | 9 | 20 | 2 | 291 | 136 | 424 | 7 |
| gb\|g2853299 | 441 | 113.0 | 2e−24 | 7 | 18 | 3 | 232 | 203 | 439 | 7 |
| gb\|g2853299 | 441 | 131.0 | 4e−30 | 8 | 21 | 3 | 290 | 49 | 343 | 7 |
| gb\|g2853299 | 441 | 134.0 | 4e−31 | 8 | 18 | 3 | 289 | 106 | 405 | 7 |
| gb\|g2853299 | 441 | 139.0 | 1e−32 | 10 | 20 | 4 | 289 | 75 | 362 | 7 |
| gb\|g2853299 | 441 | 130.0 | 6e−30 | 8 | 21 | 5 | 291 | 97 | 397 | 7 |
| gb\|g2853299 | 441 | 134.0 | 7e−31 | 5 | 21 | 5 | 273 | 168 | 439 | 7 |
| gb\|g2853299 | 441 | 123.0 | 1e−27 | 10 | 24 | 13 | 289 | 18 | 301 | 7 |
| gb\|g3876860 | 1805 | 71.8 | 4e−12 | 11 | 23 | 1 | 282 | 1003 | 1285 | 7 |
| gb\|g3876860 | 1805 | 57.0 | 1e−07 | 6 | 18 | 2 | 250 | 1129 | 1380 | 7 |
| gb\|g3876860 | 1805 | 71.0 | 7e−12 | 9 | 23 | 2 | 291 | 505 | 813 | 7 |
| gb\|g3876860 | 1805 | 51.1 | 7e−06 | 9 | 20 | 3 | 264 | 1176 | 1480 | 7 |
| gb\|g3876860 | 1805 | 70.6 | 1e−11 | 9 | 21 | 3 | 289 | 740 | 1032 | 7 |
| gb\|g3876860 | 1805 | 85.8 | 3e−16 | 10 | 17 | 4 | 291 | 836 | 1161 | 7 |
| gb\|g3876860 | 1805 | 65.5 | 3e−10 | 11 | 22 | 5 | 285 | 1010 | 1314 | 7 |
| gb\|g3876860 | 1805 | 68.3 | 5e−11 | 10 | 17 | 5 | 292 | 929 | 1260 | 7 |
| gb\|g3876860 | 1805 | 76.0 | 2e−13 | 12 | 21 | 5 | 291 | 906 | 1216 | 7 |
| gb\|g3876860 | 1805 | 57.4 | 9e−08 | 10 | 19 | 6 | 291 | 385 | 742 | 7 |
| gb\|g3876860 | 1805 | 74.9 | 5e−13 | 7 | 15 | 9 | 289 | 716 | 1023 | 7 |
| gb\|g3876860 | 1805 | 82.7 | 2e−15 | 12 | 20 | 10 | 288 | 785 | 1077 | 7 |
| gb\|g3876861 | 1797 | 71.8 | 4e−12 | 11 | 23 | 1 | 282 | 1003 | 1285 | 7 |
| gb\|g3876861 | 1797 | 57.0 | 1e−07 | 6 | 18 | 2 | 250 | 1129 | 1380 | 7 |
| gb\|g3876861 | 1797 | 71.0 | 7e−12 | 9 | 23 | 2 | 291 | 505 | 813 | 7 |
| gb\|g3876861 | 1797 | 50.3 | 1e−05 | 10 | 21 | 3 | 248 | 1176 | 1464 | 7 |
| gb\|g3876861 | 1797 | 70.6 | 1e−11 | 9 | 21 | 3 | 289 | 740 | 1032 | 7 |
| gb\|g3876861 | 1797 | 85.8 | 3e−16 | 10 | 17 | 4 | 291 | 836 | 1161 | 7 |
| gb\|g3876861 | 1797 | 65.5 | 3e−10 | 11 | 22 | 5 | 285 | 1010 | 1314 | 7 |
| gb\|g3876861 | 1797 | 68.3 | 3e−10 | 10 | 17 | 5 | 292 | 929 | 1260 | 7 |
| gb\|g3876861 | 1797 | 76.0 | 2e−13 | 12 | 21 | 5 | 291 | 906 | 1216 | 7 |
| gb\|g3876861 | 1797 | 57.4 | 9e−08 | 10 | 19 | 6 | 291 | 385 | 742 | 7 |
| gb\|g3876861 | 1797 | 74.9 | 5e−13 | 7 | 15 | 9 | 289 | 716 | 1023 | 7 |
| gb\|g3876861 | 1797 | 82.7 | 2e−15 | 12 | 20 | 10 | 288 | 785 | 1077 | 7 |
| gb\|g435535 | 235 | 74.9 | 5e−13 | 7 | 18 | 1 | 133 | 81 | 224 | 7 |
| gb\|g435535 | 235 | 92.4 | 3e−18 | 7 | 16 | 10 | 165 | 54 | 224 | 7 |
| gb\|g435535 | 235 | 93.2 | 2e−18 | 8 | 20 | 11 | 171 | 54 | 224 | 7 |
| gb\|g435535 | 235 | 94.4 | 7e−19 | 8 | 16 | 23 | 185 | 54 | 224 | 7 |
| gb\|g435535 | 235 | 93.6 | 1e−18 | 7 | 18 | 43 | 200 | 54 | 224 | 7 |
| gb\|g435535 | 235 | 96.7 | 1e−19 | 6 | 19 | 70 | 231 | 54 | 224 | 7 |
| gb\|g435535 | 235 | 99.0 | 3e−20 | 10 | 18 | 98 | 259 | 54 | 224 | 7 |
| gb\|g435535 | 235 | 97.1 | 1e−19 | 5 | 16 | 137 | 291 | 54 | 224 | 7 |
| gb\|g160473 | 280 | 53.9 | 1e−06 | 7 | 18 | 2 | 151 | 63 | 213 | 8 |
| gb\|g160473 | 280 | 59.3 | 2e−08 | 8 | 20 | 3 | 150 | 42 | 187 | 8 |
| gb\|g160473 | 280 | 67.9 | 6e−11 | 6 | 21 | 7 | 218 | 15 | 213 | 8 |
| gb\|g160473 | 280 | 60.5 | 1e−08 | 6 | 22 | 21 | 222 | 47 | 250 | 8 |
| gb\|g160473 | 280 | 91.3 | 6e−18 | 5 | 25 | 91 | 259 | 24 | 190 | 8 |
| gb\|g160473 | 280 | 67.1 | 1e−10 | 6 | 19 | 140 | 286 | 42 | 187 | 8 |
| gb\|g160473 | 280 | 49.6 | 2e−05 | 5 | 22 | 156 | 290 | 16 | 150 | 8 |
| gb\|g160473 | 280 | 69.5 | 2e−11 | 7 | 20 | 165 | 290 | 48 | 177 | 8 |
| gb\|g2853297 | 338 | 144.0 | 6e−34 | 8 | 17 | 1 | 291 | 5 | 321 | 8 |
| gb\|g2853297 | 338 | 112.0 | 3e−24 | 6 | 18 | 2 | 231 | 111 | 338 | 8 |
| gb\|g2853297 | 338 | 128.0 | 3e−29 | 9 | 20 | 2 | 255 | 61 | 334 | 8 |
| gb\|g2853297 | 338 | 73.8 | 1e−12 | 7 | 15 | 115 | 290 | 1 | 185 | 8 |
| gb\|g2853299 | 441 | 133.0 | 1e−30 | 7 | 20 | 1 | 292 | 28 | 330 | 8 |
| gb\|g2853299 | 441 | 141.0 | 3e−33 | 8 | 19 | 1 | 281 | 139 | 437 | 8 |
| gb\|g2853299 | 441 | 125.0 | 4e−28 | 10 | 19 | 2 | 241 | 204 | 439 | 8 |
| gb\|g2853299 | 441 | 143.0 | 1e−33 | 8 | 19 | 2 | 291 | 112 | 414 | 8 |
| gb\|g2853299 | 441 | 146.0 | 1e−34 | 8 | 19 | 2 | 291 | 136 | 433 | 8 |
| gb\|g2853299 | 441 | 147.0 | 7e−35 | 7 | 20 | 3 | 290 | 151 | 437 | 8 |
| gb\|g2853299 | 441 | 138.0 | 5e−32 | 9 | 21 | 4 | 291 | 104 | 397 | 8 |
| gb\|g2853299 | 441 | 140.0 | 7e−33 | 5 | 21 | 4 | 275 | 168 | 439 | 8 |
| gb\|g2853299 | 441 | 146.0 | 1e−34 | 8 | 18 | 4 | 289 | 75 | 363 | 8 |
| gb\|g2853299 | 441 | 143.0 | 1e−33 | 5 | 15 | 5 | 291 | 112 | 416 | 8 |
| gb\|g3876860 | 1805 | 68.3 | 5e−11 | 11 | 18 | 1 | 291 | 1040 | 1371 | 8 |
| gb\|g3876860 | 1805 | 71.4 | 5e−12 | 10 | 21 | 1 | 291 | 592 | 903 | 8 |
| gb\|g3876860 | 1805 | 72.6 | 2e−12 | 11 | 21 | 1 | 285 | 1003 | 1314 | 8 |
| gb\|g3876860 | 1805 | 105.0 | 3e−22 | 11 | 20 | 2 | 290 | 864 | 1159 | 8 |
| gb\|g3876860 | 1805 | 61.3 | 6e−09 | 8 | 17 | 2 | 274 | 1103 | 1380 | 8 |
| gb\|g3876860 | 1805 | 68.3 | 5e−11 | 7 | 20 | 2 | 291 | 505 | 813 | 8 |
| gb\|g3876860 | 1805 | 73.4 | 1e−12 | 9 | 18 | 5 | 291 | 727 | 1029 | 8 |
| gb\|g3876860 | 1805 | 81.9 | 4e−15 | 11 | 20 | 5 | 291 | 906 | 1216 | 8 |
| gb\|g3876860 | 1805 | 70.2 | 1e−11 | 8 | 17 | 6 | 290 | 978 | 1273 | 8 |
| gb\|g3876860 | 1805 | 83.9 | 1e−15 | 8 | 15 | 8 | 288 | 800 | 1087 | 8 |
| gb\|g3876860 | 1805 | 57.8 | 7e−08 | 13 | 22 | 67 | 288 | 462 | 701 | 8 |
| gb\|g3876861 | 1797 | 68.3 | 5e−11 | 11 | 18 | 1 | 291 | 1040 | 1371 | 8 |
| gb\|g3876861 | 1797 | 71.4 | 5e−12 | 10 | 21 | 1 | 291 | 592 | 903 | 8 |
| gb\|g3876861 | 1797 | 72.6 | 2e−12 | 11 | 21 | 1 | 285 | 1003 | 1314 | 8 |
| gb\|g3876861 | 1797 | 105.0 | 3e−22 | 11 | 20 | 2 | 290 | 864 | 1159 | 8 |
| gb\|g3876861 | 1797 | 61.3 | 6e−09 | 8 | 17 | 2 | 274 | 1103 | 1380 | 8 |
| gb\|g3876861 | 1797 | 68.3 | 5e−11 | 7 | 20 | 2 | 291 | 505 | 813 | 8 |
| gb\|g3876861 | 1797 | 73.4 | 1e−12 | 9 | 18 | 2 | 291 | 727 | 1029 | 8 |
| gb\|g3876861 | 1797 | 81.9 | 4e−15 | 11 | 20 | 5 | 291 | 906 | 1216 | 8 |
| gb\|g3876861 | 1797 | 70.2 | 1e−11 | 8 | 17 | 6 | 290 | 978 | 1273 | 8 |
| gb\|g3876861 | 1797 | 83.9 | 1e−15 | 8 | 15 | 8 | 288 | 800 | 1087 | 8 |
| gb\|g3876861 | 1797 | 57.8 | 7e−08 | 13 | 22 | 67 | 288 | 462 | 701 | 8 |
| gb\|g435535 | 235 | 83.9 | 1e−15 | 6 | 16 | 1 | 133 | 81 | 224 | 8 |
| gb\|g435535 | 235 | 96.3 | 2e−19 | 7 | 19 | 2 | 171 | 53 | 224 | 8 |
| gb\|g435535 | 235 | 99.5 | 2e−20 | 6 | 16 | 35 | 191 | 54 | 224 | 8 |
| gb\|g435535 | 235 | 98.7 | 3e−20 | 7 | 21 | 45 | 200 | 54 | 224 | 8 |
| gb\|g435535 | 235 | 98.3 | 4e−20 | 11 | 20 | 59 | 215 | 54 | 224 | 8 |
| gb\|g435535 | 235 | 97.9 | 6e−20 | 6 | 15 | 82 | 241 | 54 | 224 | 8 |
| gb\|g435535 | 235 | 103.0 | 1e−21 | 10 | 17 | 98 | 258 | 54 | 224 | 8 |
| gb\|g435535 | 235 | 104.0 | 5e−22 | 4 | 16 | 132 | 291 | 54 | 224 | 8 |

Below are shown the results after the clustering procedure performed according to the present invention.

| | | | | Best | | | | | | | Best | First | First | Last |
|---|---|---|---|---|---|---|---|---|---|---|---|---|---|---|
| Sequence | Cluster | Length | Bit-Score | E-Value | ID | +ve | From | To | From | To | Iteration | Iteration | E−Value | Iteration |
| gb\|g2853297 | 1 | 338 | 144.0 | 6e−34 | 8 | 17 | 1 | 292 | 1 | 338 | 8 | 5 | 1e−14 | 8 |
| gb\|g2853299 | 1 | 441 | 147.0 | 7e−35 | 7 | 20 | 1 | 292 | 18 | 439 | 8 | 5 | 4e−16 | 8 |
| gb\|g3876860 | 1 | 1805 | 105.0 | 3e−22 | 11 | 20 | 1 | 292 | 385 | 1380 | 8 | 6 | 2e−09 | 8 |
| gb\|g3876860 | 2 | 1805 | 51.1 | 7e−06 | 9 | 20 | 3 | 264 | 1176 | 1480 | 7 | 7 | 7e−06 | 7 |
| gb\|g3876861 | 1 | 1797 | 105.0 | 3e−22 | 11 | 20 | 1 | 292 | 385 | 1380 | 8 | 6 | 2e−09 | 8 |
| gb\|g3876861 | 2 | 1797 | 50.3 | 1e−05 | 10 | 21 | 3 | 248 | 1176 | 1464 | 7 | 7 | 1e−09 | 7 |
| gb\|g435535 | 1 | 235 | 99.5 | 2e−20 | 6 | 16 | 1 | 215 | 53 | 224 | 8 | 6 | 3e−07 | 8 |
| gb\|g435535 | 2 | 235 | 97.9 | 6e−20 | 6 | 15 | 52 | 241 | 54 | 224 | 8 | 6 | 6e−14 | 8 |
| gb\|g435535 | 3 | 235 | 103.0 | 1e−21 | 10 | 17 | 98 | 259 | 54 | 224 | 8 | 6 | 8e−16 | 8 |
| gb\|g435535 | 4 | 235 | 104.0 | 5e−22 | 4 | 16 | 131 | 291 | 54 | 224 | 8 | 6 | 9e−15 | 8 |
| gb\|g160473 | 1 | 280 | 91.3 | 6e−18 | 5 | 25 | 2 | 291 | 15 | 250 | 8 | 7 | 2e−07 | 8 |

The number of alignments has been reduced from 153 to 11 on this example.

1. A method for reducing the number of results generated by the alignment of a query sequence against a target sequence by an alignment algorithm, said method comprising the step of combining two or more alignment results into a single alignment result for each specific region of sequence alignment identified between query and target sequences.

2. A computer-implemented method for reducing the number of results generated by the alignment of a query sequence against a target sequence by an iterative alignment algorithm, said method comprising the steps of:
(a) extracting said alignment results;
(b) combining two or more alignment results into a single alignment result for each specific region of sequence alignment identified between query and target sequences; and
(c) outputting said single result.

3. A method according to claim 1 or claim 2, wherein if a first alignment between a query sequence A at positions [F_{A}, T_{A}] and a target sequence B at positions [F_{B}, T_{B}] is represented graphically with the horizontal axis representing the residue numbers from sequence A, and the vertical axis representing the residue numbers from sequence B, such that a rectangular region marked by co-ordinates [F_{A}, F_{B}], [T_{A}, F_{B}], [T_{B}, F_{A}], and [T_{A}, T_{B}] represents a first region of alignment, and a second alignment between the query sequence at positions [F′_{A}, T′_{A}] and the target sequence at positions [F′_{B}, T′_{B}] is represented graphically such that a rectangular region marked by co-ordinates [F′_{A}, F′_{B}], [T′_{A}, F′_{B}], [T′_{B}, F′_{A}], and [T′_{A}, T′_{B}] represents a second region of alignment, then the first and second alignments are combined if there is a significant region of intersection between the two regions of alignment.

4. A method according to claim 3, wherein a significant region of intersection is defined as one region of alignment being greater than or equal to 90% of the area of the smaller of the two regions of alignment.

5. A method according to any one of the preceding claims that is a computer-implemented method.

6. A method according to any one of the preceding claims, wherein said combining step is repeated for every alignment that is generated by an alignment algorithm.

7. A method according to claim 6, wherein said alignment algorithm is an iterative alignment algorithm.

8. A method according to claim 7, wherein said iterative alignment algorithm is based on the Position-Specific Iteration Basic Local Alignment of Sequences Tool (PSI-BLAST) algorithm.

9. A method according to any one of the preceding claims, wherein a graph subset construction algorithm tool is used to compare the alignments.

10. A method according to any one of claims 2-9, wherein said combining step b) comprises the sequential steps of:
i. combining alignment regions in which one alignment region subsumes another; and
ii. combining alignment regions that only partially overlap.

11. A method according to any one of the preceding claims, wherein the lowest and highest iteration/E-value pair present in the two alignments, the lowest E value achieved by either of the two alignments and the iteration number in which this lowest E-value occurred are stored in the combined alignment.

12. A computer apparatus adapted to reduce the number of results generated by the alignment of a query sequence against a target sequence, said apparatus comprising:
a processor means;
a memory means; and
computer software stored in said memory and adapted to reduce the number of results generated by the alignment of a query sequence against a target sequence using a method according to any one of claims 1 to 11 and output an alignment result.

13. A computer system adapted to reduce the number of results generated by the alignment of a query sequence against a target sequence, wherein said system performs a method according to any one of the preceding claims and outputs an alignment result.

14. A computer system according to claim 13, comprising:
a central processing unit;
an input device for inputting requests;
an output device;
a memory;
at least one bus connecting the central processing unit, the memory, the input device and the output device;
the memory storing a module that is configured so that upon receiving a request to align a query sequence with a target sequence, it performs a method according to any one of claims 1 to 11.

15. A computer program product for use in conjunction with a computer, said computer program comprising a computer readable storage medium and a computer program mechanism embedded therein, the computer program mechanism comprising a module that is configured so that upon receiving a request to align two or more sequences together, it performs a method as recited in any one of claims 1 to 11 and outputs an alignment result.
